SEQUENCE LISTING (10 sequences)

SEQ ID NO: 1 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 2 - protein, 258 aa, Papio hamadryas
MWFLVLCLALSLGGTGAAPPIQSRIVGGWECSQPWQAALYHFSTFQCGGILVHPQWVLTAAHCIGDNYQLWLGRHNLFDDEDTAQFVHVSESFPHPCFNMSLLKNHTRQADEDYSHDLMLLRLTQPAEITDAVQVVELPTQEPEVGSTCLASGWGSIEPENFSYPDDLQCVDLKILPNDKCAKAHTQKVTEFMLCAGHLEGGKDTCVGDSGGPLTCDGVLQGVTSWGYIPCGSPNKPAVFVRVLSYVKWIEDTIAENS

SEQ ID NO: 3 - protein, 257 aa, Macaca fascicularis
MWFLVLCLALSLGGTGRAPPIQSRIVGGWECSQPWQAALYHFSTFQCGGILVHPQWVLTAAHCISDNYQLWLGRHNLFDDEDTAQFVHVSESFPHPGFNMSLLKNHTRQADDYSHDLMLLRLTQPAEITDAVQVVELPTQEPEVGSTCLASGWGSIEPENFSFPDDLQCVDLEILPNDECAKAHTQKVTEFMLCAGHLEGGKDTCVGDSGGPLTCDGVLQGVTSWGYIPCGSPNKPAVFVKVLSYVKWIEDTIAENS

SEQ ID NO: 4 - protein, 261 aa, Saguinus oedipus
MWFLVLCLALSLGGTGAVPPIQSRIVGGWDCKQHSQPWQAALYHYSTFQCGGVLVHPQWVLTAAHCISDHYQLWLGRHDLFENEDTAQFVFVSKSFPHPDFNMSLLKNHTRLPGEDYSHDLMLLQLKQPVQITDAVKVVELPTEGIEVGSTCLASGWGSIKPEKFSFPDILQCVDLKILPNDECDKAHAQKVTEFMLCAGPLKDGQDTCVGDSGGPLTCDGVLQGIISWGYIPCGSPNKPSVFVRVLSYVKWIKDTIADNS

SEQ ID NO: 5 - protein, 261 aa, Canis lupus familiaris
MWFLVLCLALSLAGTGAAPPVQSRIIGGWDCTKNSQPWQAALYHYSKFQCGGVLVHPEWVVTAAHCINDNYQLWLGRYNLFEHEDTAQFVQVRESFPHPEFNLSLLKNHTRLPEEDYSHDIMLLRLAEPAQITDAVRVLDLPTQEPQVGSTCYASGWGSIEPDKFIYPDDLQCVDLELLSNDICANAHSQKVTEFMLCAGHLEGGKDTCVGDSGGPLICDGVLQGITSWGHVPCGSPNMPAVYTKVISHLEWIKETMTANP

SEQ ID NO: 6 - protein, 261 aa, Ovis aries
MWFPVLCLALSLAGTGAVPPVQSRIVGGQECEKHSQPWQVAIYHFSTFQCGGVLVAPQWVLTAAHCKSENYQVWLGRHNLFEDEDTAQFAGVSEDFPNPGFNLSLLENHTRQPGEDYSHDLMLLRLQEPVQLTQDVQVLGLPTKEPQLGTTCYASGWGSVKPDEFSYPDDLQCVDLTLLPNEKCATAHPQEVTDCMLCAGHLEGGKDTCVGDSGGPLICEGMLQGITSWGHIPCGTPNKPSVYTKVIVYLDWINKTMTDNP

SEQ ID NO: 7 - protein, 261 aa, Oryctolagus cuniculus
MWLPVLCLALSLGGTGAAPPLQSRIIGGWVCGKNSQPWQAALYHYSNFQCGGVLVHPQWVLTAAHCFSDNYQLWLGRHNLFEDEAEAQFIQVSGSFPHPRFNLSLLENQTRGPGEDYSHDLMLLKLARPVQLTNAVRVLELPTQEPQVGTSCLASGWGSITPIKFTYPDELQCVDLSILANSECDKAHAQMVTECMLCAGHLEGGRDTCVGDSGGPLVCNNELQGITSWGHVPCGSPNKPAVFTKVLSYVEWIRNTIANNP

SEQ ID NO: 8 - protein, 261 aa, Bos taurus
MWFPVLCLALSLAGTGAVFPIQSRIVGGQECEKHSQPWQVAIYHFSTFQCGGVLVAPQWVLTAAHCKSDNYQVWLGRHNLFEDEDTAQFAGVSEDFPNPGFNLSLLENHTRHPGEDYSHDLMLLRLQEPVQLTQNVQVLGLPTKEPQLGTTCYASGWGSVKPDEFSYPDDLQCVDLTLLPNEKCATAHPQEVTEWMLCAGHLEGGKDTCVGDSGGPLICEGMLQGITSWGHIPCGTPNKPSVYTKVILYLDWINKTMTDNP

SEQ ID NO: 9 - protein, 261 aa, Equus ferus caballus
MWLPVLCLALSLVGTGAAPPIQSRIIGGWECKNHSKPWQAAVYHYSSFQCGGVLVDPQWVLTAAHCKGDYYQIWLGRHNLFEDEDTAQFFLVAKSFPHPDFNMSLLENNNRLPGEDYSHDLMLLQVEQPDQITVAVQVLALPTQEPVLGSTCYASGWGSIEPDKFTYPDELRCVDLTLLSNDVCDNAHSQNVTEYMLCAGHLEGGKDTCVGDSGGPLICDGVFQGVTSWGHIPCGRPNKPAVYTKLIPHVQWIQDTIAANP

SEQ ID NO: 10 - protein, 263 aa, sus scrofa
MWSLVMRLALSLAGTGAAPPIQSRIIGGRECEKDSHPWQVAIYHYSSFQCGGVLVDPKWVLTAAHCKNDNYQVWLGRHNLFENEVTAQFFGVTADFPHPGFNLSLLKNHTKADGKDYSHDLMLLRLQSPAKITDAVKVLELPTQEPELGSTCQASGWGSIEPGPDDFEFPDEIQCVELTLLQNTFCADAHPDKVTESMLCAGYLPGGKDTCMGDSGGPLICNGMWQGITSWGHTPCGSANKPSIYTKLIFYLDWINDTITENP

(19) United States
(12) Patent Application Publication
(10) Pub. No.: US 2012/0225051 A1
(43) Pub. Date: Sep. 6, 2012

(54) TISSUE KALLIKREIN FOR THE TREATMENT OF HUNTINGTON'S DISEASE

(75) Inventors: Mark Williams, Winnipeg (CA); Matthew Charles, Winnipeg (CA)

(73) Assignee: Sanomune Inc., Winnipeg, MB (CA)

(21) Appl. No.: 13/265,357

(22) PCT Filed: Apr. 22, 2010
(86) PCT No.: PCT/CA2010/000575

§ 371 Date: Jan. 6, 2012

Related U.S. Application Data
(60) Provisional application No. 61/171,579, filed on Apr. 22, 2009.

(51) Int. Cl. A61K 38/48; A61P 25/00

(52) U.S. Cl. 424/94.64

(57) ABSTRACT

This invention relates to methods of treating the prodrome and adult onset stage of Huntington's disease or symptoms thereof, and or Juvenile Huntington's disease symptoms thereof. Methods of the invention include administering a therapeutically effective amount of tissue kallikrein, variants or active fragments thereof. The invention further relates to pharmaceutical compositions comprising a therapeutically effective amount of tissue kallikrein, variants or active fragments thereof formulated for oral or intranasal administration.

### CROSS REFERENCE TO RELATED APPLICATIONS

This application claims the benefit of and priority to U.S. Patent Application No. 61/171,579, filed Apr. 22, 2009, under the title USE OF KALLIKREIN TO TREAT HUNTINGTON'S DISEASE.

The content of the above patent application is hereby expressly incorporated by reference into the detailed description hereof.

### FIELD OF THE INVENTION

The present invention relates to methods of treating the prodrome and onset of adult Huntington's disease, juvenile Huntington's disease, and associated conditions.

### BACKGROUND OF THE INVENTION

Huntington's Disease (HD) is polygutamine neurodegenerative disorder resulting from the unstable expansion of CAG trinucleotide repeats at the 5′ end of HD gene (The Huntington's Disease Collaborative Research Group, 1993). This mutation results in the formation of a mutant huntingtin protein that bears a stretch of glutamate residues at the N-terminal of the protein. HD is an autosomally inherited disease in which the mutant HD gene allele is unstable and CAG repeats can be increased or decreased. The number of CAG repeats is the main determinant of disease severity (Butterworth et al., 1998) where 35-39 repeats are associated with late onset, 40-50 for the common adult onset and longer repeats for juvenile onset (The Huntington's Disease Collaborative Research Group, 1993).

The disease has a prevalence of 5-10 cases per 100,000 individuals, with about 30,000 affected individuals in North America (Hersch and Rosas, 2008). The average age of clinical onset is about 37 years of age (adult onset), and approximately 10-15 years after onset patients generally die from heart failure or aspiration pneumonia. The underlying processes which causes adult onset HD are thought to be active 10-20 years before HD manifests unequivocally and is clinically diagnosed by the presence of a movement disorder. The transition from the HD premanifest prodrome to diagnosis (manifest HD) is seen by progressive neuronal hypometabolism and neurodegeneration (neuronal cell loss/brain atrophy/volume loss) of the striatum, cerebral cortex, and white matter areas of the brain as detected by PET and MRI (Ciarmiello et al., 2006; Harris et al., 1999). During the prodrome stage, gradual changes in motor skill, behavioural, metabolic and psychological areas are seen.

HD is classified as a movement disorder which in its earliest stages causes minor motor skill abnormalities including restlessness, abnormal eye movements, hyperreflexia, impaired finger tapping, rapid alternating hand movements, and mild dysarthria (Aubeeluck and Wilson, 2008). In the mid-stage of the disease, involuntary motor abnormalities become more pronounced including chorea (rapid, ceaseless movements). Voluntary motor impairments will include dysphagia (swallowing), dysarthria, and gait disturbances. At the end stage of the disease, bradykinesia, hypokinesia, rigidity, and dystonia dominate and replace chorea movements. By this stage, many HD patients also experience muscle wasting, dehydration and weight loss due to decreased exercise and difficulty swallowing food thus increased feeding, fluids and assisted exercise are necessary with the assistance of a caregiver. Non-motor symptoms are also present in HD, including cognitive deficits in concentration, organization, spatial perception and memory skills (dementia) and non-cognitive psychiatric deficits including depression (low energy, sleep disturbances), personality changes (irritability, low energy, apathy anhedonia) and bipolar disorder (delusions, hallucinations, paranoia).

Current treatment is focused on preventing symptoms, including antipsychotics (haloperidol, chlorpromazine, olanzapine), antidepressants (fluoxetine, sertraline hydrochloride, nortriptyline), tranquilizers (benzodiazepines, paroxetine, venlafaxin, beta-blockers), mood-stabilizers (lithium, valproate, carbamazepine), and Botulinum toxin (for muscle contractions and jaw clenching) medications.

A hallmark of neurodegeneration in HD is the atrophy of the striatum region of the brain (Vonsattel et al., 1985). Within the striatum, a particular group of neurons called medium spiny neurons is most susceptible to degradation. These neurons are GABAergic which produce the inhibitory neurotransmitter γ-aminobutyric acid (GABA) and thus are responsible from controlling movements of the body, limbs, and eyes (Gil and Rego, 2008). They receive dopamingeric and glutamatergic excitatory inputs from the substantia nigar pars compacta and cerebral cortex, respectively. Thus it is believed that the neurodegenerative loss of these medium spiny neurons inhibitory input leads to the uncontrolled movements seen in HD patients.

The mutant huntingtin protein, with an expanded N-terminal polyglutamate tail, is thought to be the primary cause of the neurodegeneration. The normal cellular function of wild type huntingtin is still not completely clear, however, appears to involved in multiple cellular processes and pathways through various protein-protein interactions such as HAP1/dynactin (vesicular transport of BDNF)(Gauthier et al., 2004), HIP1 (cytoskeleton and endocytosis)(Kaltenbach et al., 2007; Waelter et al., 2001), HIP2 (ubiquitin mediated protein degradation by protease)(de Pril et al., 2007), GAPDH (glycolysis)(Kaltenbach et al., 2007), PSD95 (anchoring of NMDA receptor at postsynaptic membrane)(Sun et al., 2001), REST-NRSF transcription factor complex in cytoplasm (allows for BDNF transcription, neuron cell survival)(Zuccato et al., 2003), preventing activation of caspase-3, caspase-6, caspase-8 (anti-apoptosis, neuron cell survival)(Gil and Rego, 2008).

However, the toxicity of mutant huntingtin is believed to be mediated through multiple mutant gain of and wild type loss of huntingtin functions which result cellular dysfunction and apoptosis.

Mutant huntingtin protein can block ubiquitin-proteasome degradation of misfolded proteins leading to apoptosis (Jana et al., 2001). The mutant protein can adversely affect histone acetylation and transcription by abnormal interaction with transcription factors (e.g. CBP, CBP-TAFII130, Sp1, NFκB)(Roze et al., 2008). It can no longer bind REST/NRSF repressor in cytoplasm leading to the inhibition of BDNF expression, an important neuronal survival factor (Zuccato et al., 2003) and activation of caspase-8 normally inhibited by wild type huntingtin (Gervais et al., 2002). Mutant huntingtin can also suppress the activity of PGC-1α (Cui et al., 2006), leading to decreased transcription of ROS-detoxifying enzymes and mitochondrial biogenesis resulting in mitochondria dysfunction (less ATP, increased ROS oxidative stress).

Mutant huntingtin also disrupts axonal transport leading to dysfunction at the synapse. This includes lost ability to interact with HAP1 for axonal transport and depleting proteins involved in vesicle recycling and receptor endocytosis (e.g. complexin II, synaptobrevin-2, rabphilin A) (Gil and Rego, 2008). Lack of interactions between mutant huntingtin and PSD-95 (Sun et al., 2001) leads to increased trafficking of NMDA (NR2B subunit) receptors to the synapse which leads to glutamate excitotoxcity through excessive Ca2+ influx and mitochondrial dysfunction triggering apoptosis. Excitotoxicty is enhanced by dopamine (Tang et al., 2007), a neurotransmitter found at high concentrations in the striatum.

None of the current treatments used in HD prevent the onset or progression of HD caused by neurodegeneration, they only temporarily alleviate some of the symptoms of the disease. As there is no proven neuroprotective therapy, ongoing drug development is targeted towards the various types of cellular dysfunction mutant huntingtin causes. This includes the use of antioxidants (CoQ10, Ethyl-EPA, Creatine, cysteamine, indole-3-propionic acid, N-acetylcysteine) to prevent mitochondrial dysfunction and improve cellular energy metabolism, anti-apoptosis drugs to prevent the triggering of programmed cell death (minocycline, tauroursodexoycholic acid, methazolamide, caspase 6 inhibitor,), blocking glutamate excitotoxicity (memantine, remacemide), mitochondrial stabilization (dimebon), blocking the effects of dopamine (tetrabenazine), neurotrophic growth factors to promote neurogenesis and survival (FGF2, Neurturin), prevention of mutant Huntingtin allele expression (RNAi, antisense), increase transcription and increase expression of such proteins as BDNF for survival (cysteamine, HDAC inhibitors, Citalopram), induction of autophagy to clear huntingtin aggregates (verapamil, clonidine, rampamycin) or use of restorative stem cells.

### SUMMARY OF THE INVENTION

The present invention includes tissue kallikrein or a variant or active fragment thereof in treating the prodrome and onset stage of Huntington's disease or symptoms thereof and juvenile Huntington's disease or symptoms thereof.

In one aspect, a method of treating a patient having (a) Huntington's disease or symptoms thereof or (b) juvenile Huntington's disease or symptoms thereof includes administering a therapeutically effective amount of tissue kallikrein or a variant or active fragment thereof to said patient.

In one aspect tissue kallikrein can be an isolated form, a synthetic form, or a recombinant form of KLK1.

In another aspect, isolated KLK1 can be human KLK1 (SEQ ID NO: 1)

In another aspect, isolated KLK1 can be hamadryas baboon KLK1 (SEQ ID NO. 2), crab eating macaque KLK1 (SEQ ID NO. 3), cotton top tamarin KLK1 (SEQ ID NO. 4), dog KLK1 (SEQ ID NO. 5), sheep KLK1 (SEQ ID NO. 6), rabbit KLK1 (SEQ ID NO. 7), bovine KLK1 (SEQ ID NO. 8), horse KLK1 (SEQ ID NO. 9), or pig KLK1 (SEQ ID NO. 10), or a variant or active fragment thereof.

In an embodiment of the invention, tissue kallikrein, or a variant or active fragment thereof, is administered concurrently with a second therapeutic compound useful in treating Huntington's disease or juvenile Huntington's disease.

In another aspect, a pharmaceutical composition comprises about 1 to about 1000 IU per day of tissue kallikrein, or a variant or active fragment thereof, and a pharmaceutically acceptable excipient formulated for oral administration.

In another aspect, a pharmaceutical composition comprises about 0.001 to about 5000 IU per dosage frequency, or a variant or active fragment thereof, and a pharmaceutically acceptable excipient formulated for intranasal administration.

In an embodiment, pharmaceutical composition according to the invention includes a tissue kallikrein, or a variant or active fragment thereof combined with an adjuvant.

In a further embodiment of the invention, the adjuvant is an emulsifier.

In a further embodiment, the pharmaceutical composition according to the invention comprises a tissue kallikrein, or a variant or active fragment thereof combined with lipophillic micelles.

In a further embodiment, the pharmaceutical composition according to the invention further comprises a second therapeutic compound useful in treating Huntington's disease.

Another aspect of the invention includes use of a tissue kallikrein, or a variant or active fragment thereof for the preparation of a medicament useful for treating: (a) Huntington's disease or symptoms thereof, or (b) juvenile Huntington's disease or symptoms thereof.

Another aspect of the invention includes use of a therapeutically effective amount of tissue kallikrein, or a variant or active fragment thereof for the treatment of: (a) Huntington's disease or symptoms thereof, or (b) juvenile Huntington's disease or symptoms thereof.

In an embodiment of the invention, use of tissue kallikrein, or a variant or active fragment thereof, further comprises concurrent use of a second therapeutic compound useful in treating prodrome and onset stage of Huntington's disease and Juvenile Huntington's disease.

In a further embodiment of the invention, a second therapeutic compound can be a nutritional supplement, an anti-oxidant, an anti-apoptotic compound, a NDMA receptor antagonist, a mitochondrial stabilizer, a dopamine blocker, a BDNF inducer, an inducer of transcription, a blocker of mutant huntingtin allele expression, a neurotrophic factor, inducer of autophagy,or restorative neural stem cell therapy, an antipsychotic, an antidepressant, a tranquilizer, a mood-stabilizer, or a muscle relaxant. I

In further embodiments of the invention, a nutritional supplement can be CoQ10, Ethyl-EPA, or creatine.

In a further embodiment of the invention, an anti-oxidant can be cysteamine, indole-3-propionic acid, N-acetylcysteine, glutathione, or bucillamine.

In a further embodiment of the invention, an anti-apoptotic compound is selected from minocycline, tauroursodexoycholic acid, methazolamide, or caspase-6 inhibitor

In a further embodiment of the invention, a NDMA receptor antagonist can be memantine or remacemide

In a further embodiment of the invention, a mitochondrial stabilizer can be dimebon.

In a further embodiment of the invention, a dopamine blocker can be tetrabenazine

In a further embodiment of the invention, a BDNF inducer can be cysteamine or citalopram

In a further embodiment of the invention, a transcription inducer can be a HDAC inhibitor

In a further embodiment of the invention, a blocker of mutant huntingtin allele expression can be a huntingtin gene specific RNAi or antisense molecule.

In a further embodiment of the invention, a neurotrophic factor can be FGF2 or neurturin

In a further embodiment of the invention, an inducer of autophagy can be verapamil, clonidine, or rampamycin.

In a further embodiment of the invention, a restorative neural stem cell therapy can be introduction of isolated stem cells into the brain.

In a further embodiment of the invention, an antipsychotic can be haloperidol, chlorpromazine, or olanzapine.

In a further embodiment of the invention, an antidepressant can include fluoxetine, sertraline hydrochloride, or nortriptyline.

In a further embodiment of the invention, a tranquilizer can be benzodiazepines, paroxetine, venlafaxin, or a beta-blocker.

In a further embodiment of the invention, the mood-stabilizer can include lithium, valproate, or carbamazepine.

In a further embodiment of the invention, the muscle relaxant can include Botulinum toxin, diazepam, skelaxin, cariosprodol, or cyclobenzaprine.

In a further embodiment of the invention, a therapeutically effective amount of tissue kallikrein, or a variant or active fragment thereof can be administered intranasally.

In a further embodiment of the invention, a therapeutically effective dose is about 0.001 to about 5000 International Units (IU) dosage frequency.

In a further embodiment of the invention, a therapeutically effective amount of tissue kallikrein, or a variant or active fragment thereof can be administered orally.

In a further embodiment of the invention, a therapeutically effective of tissue kallikrein, or a variant or active fragment thereof can be about 0.001 to about 1000 IU per day.

In a further aspect, use of a therapeutically effective amount of tissue kallikrein, or a variant or active fragment thereof can be administered for improving oxygen uptake to the brain of a patient in need thereof.

In a further aspect, use of a therapeutically effective amount of tissue kallikrein, or a variant or active fragment thereof can be for improving blood flow to the brain of a patient in need thereof.

In a further aspect, use of a therapeutically effective amount of tissue kallikrein, or a variant or active fragment thereof can be for improving glucose uptake by the brain of a patient in need thereof.

In a further aspect, use of a therapeutically effective amount of tissue kallikrein, or a variant or active fragment thereof can be for improving volume of the brain of a patient in need thereof.

In a further aspect, use of a therapeutically effective amount of tissue kallikrein, or a variant or active fragment thereof can be for improving cellular metabolism in the brain of a patient in need thereof.

In a further embodiment of the invention, a method of treating prodrome and onset stage of Huntington's disease or juvenile Huntington's disease may be assessed and monitored by the use of standardized scales including: the Total Functional Capacity (TFC) portion of the Unified Huntington's Disease Rating Scale (UHDRS) and the mini-mental state examination (MMSE).

In a further embodiment of the invention, standardized scales include, but are not limited to, the Total Functional Capacity (TFC) portion of the Unified Huntington's Disease Rating Scale (UHDRS) and the mini-mental state examination (MMSE) or a combination thereof.

### DETAILED DESCRIPTION

### Definitions

“Tissue kallikrein” or “KLK1” is a serine protease that is primarily noted for its role in controlling hypertension through its cleavage of kininogen into lysyl-bradykinin (kallidin) (Yousef and Diamandis, 2001). As there are a large number of enzymes in the KLK family, the inventors believe that KLK1 appears to be a ubiquitous or multiple target acting enzyme, in addition to its recognized role in hypertension regulation and as such may specifically play an important role in treating the prodrome and onset stage of Huntington's disease and Juvenile Huntington's disease. As used herein, the term “tissue kallikrein” is synonymous with the following terms: callicrein, glumorin, padreatin, padutin, kallidinogenase, bradykininogenase, panceatic kallikrein, onokrein P, dilminal D, depot-Padutin, urokallikrein, or urinary kallikrein.

As described above, “kallidin” refers to lysyl-bradykinin. Kallikrein cleaves kininogen into kallidin. Kallidin can activate the bradykinin 2 receptor.

A preferred embodiment of the present invention is human tissue kallikrein precursor polypeptide (kidney/pancreas/salivary gland kallikrein) (KLK1), variant, or active fragment thereof, and has the following sequence (SEQ ID NO:1):

NP_002248 GI: 4504875 Homo sapiens KLK1_human
1-18 signal peptide
19-24 propeptide
25-262 mature peptide
(SEQ ID NO: 1)
MWFLVLCLALSLGGTGAAPPIQSRIVGGWECEQHSQPWQAALYHFSTFQC
GGILVHRQWVLTAAHCISDNYQLWLGRHNLFDDENTAQFVHVSESFPHPG
FNMSLLENHTRQADEDYSHDLMLLRLTEPADTITDAVKVVELPTEEPEVG
STCLASGWGSIEPENFSFPDDLQCVDLKILPNDECKKAHVQKVTDFMLCV
GHLEGGKDTCVGDSGGPLMCDGVLQGVTSWGYVPCGTPNKPSVAVRVLSY
VKWIEDTIAENS

A further embodiment of the present invention includes hamadryas baboon tissue kallikrein (kidney/pancreas/salivary gland kallikrein) (SEQ ID NO. 2) which has 90% sequence identity to human KLK1 (SEQ ID NO:1)

Q2877 KLK1_PAPHA
(SEQ ID NO: 2)
MWFLVLCLALSLGGTGAAPPIQSRIVGGWECSQPWQAALYHFSTFQCGGI
LVHPQWVLTAAHCIGDNYQLWLGRHNLFDDEDTAQFVHVSESFPHPCFNM
SLLKNHTRQADEDYSHDLMLLRLTQPAEITDAVQVVELPTQEPEVGSTCL
ASGWGSIEPENFSYPDDLQCVDLKILPNDKCAKAHTQKVTEFMLCAGHLE
GGKDTCVGDSGGPLTCDGVLQGVTSWGYIPCGSPNKPAVFVRVLSYVKWI
EDTIAENS

A further embodiment of the present invention includes crab eating macaque tissue kallikrein (kidney/pancreas/salivary gland kallikrein) (SEQ ID NO:3) which has 90% sequence identity to human KLK1 (SEQ ID NO:1):

Q07276-1 KLK1_MACFA
(SEQ ID NO: 3)
MWFLVLCLALSLGGTGRAPPIQSRIVGGWECSQPWQAALYHFSTFQCGGI
LVHPQWVLTAAHCISDNYQLWLGRHNLFDDEDTAQFVHVSESFPHPGFNM
SLLKNHTRQADDYSHDLMLLRLTQPAEITDAVQVVELPTQEPEVGSTCLA
SGWGSIEPENFSFPDDLQCVDLEILPNDECAKAHTQKVTEFMLCAGHLEG
GKDTCVGDSGGPLTCDGVLQGVTSWGYIPCGSPNKPAVFVKVLSYVKWIE
DTIAENS

A further embodiment of the present invention includes cotton top tamarin tissue kallikrein (kidney/pancreas/salivary gland kallikrein) (SEQ ID NO:4) which has 82% sequence identity to human KLK1 (SEQ ID NO:1):

Q9N1Q1_SAGOE
(SEQ ID NO. 4)
MWFLVLCLALSLGGTGAVPPIQSRIVGGWDCKQHSQPWQAALYHYSTFQC
GGVLVHPQWVLTAAHCISDHYQLWLGRHDLFENEDTAQFVFVSKSFPHPD
FNMSLLKNHTRLPGEDYSHDLMLLQLKQPVQITDAVKVVELPTEGIEVGS
TCLASGWGSIKPEKFSFPDILQCVDLKILPNDECDKAHAQKVTEFMLCAG
PLKDGQDTCVGDSGGPLTCDGVLQGIISWGYIPCGSPNKPSVFVRVLSYV
KWIKDTIADNS

A further embodiment of the present invention includes dog tissue kallikrein (kidney/pancreas/salivary gland kallikrein) (SEQ ID NO: 5) which has 74% sequence identity to human KLK1 (SEQ ID NO: 1):

Q29474_CANFA
(SEQ ID NO: 5)
MWFLVLCLALSLAGTGAAPPVQSRIIGGWDCTKNSQPWQAALYHYSKFQC
GGVLVHPEWVVTAAHCINDNYQLWLGRYNLFEHEDTAQFVQVRESFPHPE
FNLSLLKNHTRLPEEDYSHDIMLLRLAEPAQITDAVRVLDLPTQEPQVGS
TCYASGWGSIEPDKFIYPDDLQCVDLELLSNDICANAHSQKVTEFMLCAG
HLEGGKDTCVGDSGGPLICDGVLQGITSWGHVPCGSPNMPAVYTKVISHL
EWIKETMTANP

A further embodiment of the present invention includes sheep tissue kallikrein-1 (SEQ ID NO:6) which has 72% sequence identity to human KLK1 (SEQ ID NO:1):

A5A2L9_SHEEP
(SEQ ID NO: 6)
MWFPVLCLALSLAGTGAVPPVQSRIVGGQECEKHSQPWQVAIYHFSTFQC
GGVLVAPQWVLTAAHCKSENYQVWLGRHNLFEDEDTAQFAGVSEDFPNPG
FNLSLLENHTRQPGEDYSHDLMLLRLQEPVQLTQDVQVLGLPTKEPQLGT
TCYASGWGSVKPDEFSYPDDLQCVDLTLLPNEKCATAHPQEVTDCMLCAG
HLEGGKDTCVGDSGGPLICEGMLQGITSWGHIPCGTPNKPSVYTKVIVYL
DWINKTMTDNP

A further embodiment of the present invention includes rabbit tissue kallikrein-1 (SEQ ID NO:7) which has 73% sequence identity to human KLK1 (SEQ ID NO:1):

A5A2M0_RABIT
(SEQ ID NO: 7)
MWLPVLCLALSLGGTGAAPPLQSRIIGGWVCGKNSQPWQAALYHYSNFQC
GGVLVHPQWVLTAAHCFSDNYQLWLGRHNLFEDEAEAQFIQVSGSFPHPR
FNLSLLENQTRGPGEDYSHDLMLLKLARPVQLTNAVRVLELPTQEPQVGT
SCLASGWGSITPIKFTYPDELQCVDLSILANSECDKAHAQMVTECMLCAG
HLEGGRDTCVGDSGGPLVCNNELQGITSWGHVPCGSPNKPAVFTKVLSYV
EWIRNTIANNP

A further embodiment of the present invention includes bovine glandular kallikrein-1 precursor (SEQ ID NO:8) which has 72% sequence identity to human KLK1 (SEQ ID NO:1):

Q6H320_BOVIN
(SEQ ID NO: 8)
MWFPVLCLALSLAGTGAVFPIQSRIVGGQECEKHSQPWQVAIYHFSTFQC
GGVLVAPQWVLTAAHCKSDNYQVWLGRHNLFEDEDTAQFAGVSEDFPNPG
FNLSLLENHTRHPGEDYSHDLMLLRLQEPVQLTQNVQVLGLPTKEPQLGT
TCYASGWGSVKPDEFSYPDDLQCVDLTLLPNEKCATAHPQEVTEWMLCAG
HLEGGKDTCVGDSGGPLICEGMLQGITSWGHIPCGTPNKPSVYTKVILYL
DWINKTMTDNP

A further embodiment of the present invention includes horse glandular kallikrein-1 precursor (KLKE1) (SEQ ID NO:9) which has 70% sequence identity to human KLK1 (SEQ ID NO:1):

Q6H322_HORSE
(SEQ ID NO: 9)
MWLPVLCLALSLVGTGAAPPIQSRIIGGWECKNHSKPWQAAVYHYSSFQC
GGVLVDPQWVLTAAHCKGDYYQIWLGRHNLFEDEDTAQFFLVAKSFPHPD
FNMSLLENNNRLPGEDYSHDLMLLQVEQPDQITVAVQVLALPTQEPVLGS
TCYASGWGSIEPDKFTYPDELRCVDLTLLSNDVCDNAHSQNVTEYMLCAG
HLEGGKDTCVGDSGGPLICDGVFQGVTSWGHIPCGRPNKPAVYTKLIPHV
QWIQDTIAANP

Another preferred embodiment of the present invention includes pig glandular kallikrein-1 precursor (SEQ ID NO:10) which has 67% sequence identity to human KLK1 (SEQ ID NO:1):

NP_001001911 GI: 50054435 Sus scrofa
1-17 signal peptide
18-24 propeptide
25-263 mature peptide
>gi|50054435|ref|NP_001001911.1| kallikrein 1
[Sus scrofa]
(SEQ ID NO: 10)
MWSLVMRLALSLAGTGAAPPIQSRIIGGRECEKDSHPWQVAIYHYSSFQC
GGVLVDPKWVLTAAHCKNDNYQVWLGRHNLFENEVTAQFFGVTADFPHPG
FNLSLLKNHTKADGKDYSHDLMLLRLQSPAKITDAVKVLELPTQEPELGS
TCQASGWGSIEPGPDDFEFPDEIQCVELTLLQNTFCADAHPDKVTESMLC
AGYLPGGKDTCMGDSGGPLICNGMWQGITSWGHTPCGSANKPSIYTKLIF
YLDWINDTITENP

The term “active fragment” refers to smaller portions of the KLK1 polypeptide or a KLK1 polypeptide variant that retains the activity of the full-length KLK1 polypeptide.

A “variant” or “mutant” of a starting or reference polypeptide is a polypeptide that 1) has an amino acid sequence different from that of the starting or reference polypeptide and 2) was derived from the starting or reference polypeptide through either natural or artificial (man made) mutagenesis. Such variants include, for example, deletions from, and/or insertions into and/or substitutions of, residues within the amino acid sequence of the polypeptide of interest. A variant amino acid, in this context, refers to an amino acid different from the amino acid at the corresponding position in a starting or reference polypeptide sequence (such as that of a source antibody or antigen binding fragment). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

A “wild type” or “reference” sequence or the sequence of a “wild type” or “reference” protein/polypeptide maybe the reference sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

“Percent (%) amino acid sequence identity” with respect to polypeptides identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MegAlign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, Calif.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y,

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or MegAlign (DNASTAR) software. Appropriate parameters for measuring alignment, including any algorithms needed to achieve maximal alignment over the full-length of the sequences being compared can be determined by known methods.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z,

where W is the number of nucleotides scored as identical matches by the sequence alignment program in that program's alignment of C and D, and where Z is the total number of nucleotides in D. It will he appreciated that where the length of nucleic acid sequence C is not equal to the length of nucleic acid sequence D, the % nucleic acid sequence identity of C to D will not equal the % nucleic acid sequence identity of D to C. It is understood in the art that often, similar sequences will have similar function. For example, a sequence with 80% identity over its length with another sequence will often have similar function to the other sequence.

The term “amino acid” is used in its broadest sense and is meant to include the naturally occurring L a-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes all D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, In: The Peptides: Analysis, Synthesis, Biology, Gross and Meiehofer, Eds., Vol. 5 p 341, Academic Press, Inc, N.Y. 1983, which is incorporated herein by reference.

“Polypeptide” or “protein” refers to a peptide or protein containing two or more amino acids linked by peptide bonds, and includes peptides, oligimers, proteins, and the like. Polypeptides can contain natural, modified, or synthetic amino acids. Polypeptides can also be modified naturally, such as by post-translational processing, or chemically, such as amidation acylation, cross-linking, and the like. Examples of proteins include, but are not limited to, antibodies, enzymes, lectins and receptors; lipoproteins and lipopolypeptides; and glycoproteins and glycopolypeptides.

A “fusion protein” and a “fusion polypeptide” refers to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

The term “therapeutically effective amount” refers to an amount of a composition of this invention effective to “alleviate” or “treat” a disease or disorder in a subject or mammal. Generally, alleviation or treatment of a disease or disorder involves the lessening of one or more symptoms or medical problems associated with the disease or disorder. In some embodiments, it is an amount that improves oxygen uptake, blood flow, glucose uptake, volume and cellular metabolism mixtures thereof.

The terms “treatment” and “treating” refer to inhibiting, alleviating, and healing Huntington's disease, including, but not limited to the prodrome and onset stage of Huntington's disease, juvenile Huntington's disease, and conditions or symptoms thereof. “Treating” or “treatment” refers to therapeutic treatment, wherein the object is to prevent or slow down (lessen) the targeted pathologic condition or disorder. Treatment can be carried out by administering a therapeutically effective amount of at least one compound of the invention.

The term “improved oxygen uptake” refers to increased delivery of oxygen to the brain and cells of the brain while the term “improved blood flow” refers to an increase of blood volume circulating through the brain. Use of MRI and SPECT combined imaging, as established in the art, allows for spatial visualization of blood flow in the brain of those with prodromal HD and HD (Harris et al., 1999). A Tc-99m HMPAO tracer is used frequently to detect cerebral blood flow. Cerebral blood flow allows for oxygen uptake by surrounding tissue where blow flow is taking place. In prodromal HD and HD, cerebral blood flow within the basal ganglia, which includes the striatum (caudate and putamen), is decreased and thus decreased oxygen uptake as well. By mapping areas of blood flow and oxygen uptake deficiencies in the brain of a Huntington's disease patient, improvement can be assessed during and after treatment using an age matched non-Huntington's disease patient(s) as a control. An “increase” refers to greater blood flow or oxygen uptake in a patient after treatment compared to the blood flow or oxygen uptake in the patient before treatment.

The term “improved glucose uptake” refers to an enhanced ability of the brain to utilize glucose from the blood stream. A hallmark of Huntington's disease is a reduction of glucose uptake and metabolism in areas such as, but not limited to, the cortex and basal ganglia (hypometabolism). This marker of the prodomal stage of disease and disease onset is determined by use of PET imaging of the brain with fluorine labeled glucose contrast agent (¹⁸F-FDG PET) (Ciarmiello et al., 2006). By comparing images generated by this method before, during, and after treatment, an improvement in glucose uptake in areas of the brain in a Huntington's disease patient previously displaying a reduction of glucose uptake can be assessed while using an age-matched non-Huntington's disease control subject(s).

The term “improved volume” refers to an increase in cerebral volume as measured by MRI. Volumetric changes (decreases) in grey matter basal ganglia (including the striatum), cortex, and brain white matter are seen in prodrome and disease onset stages of Huntington's disease. Brain tissue atrophy of those with HD can be measured by comparing brain volume MRI scans with age-matched non-HD control subjects and determining volume differences (Ciarmiello et al., 2006). An improvement in volume in areas of the brain typically reduced by Huntington's disease after treatment can be assessed by comparing with before and after treatment MRI images of those with HD.

The term “improved cellular metabolism” refers to changes in levels of metabolite ratios as measured by MRS, which reflect a return to normal cellular metabolism in the brain. Certain metabolite ratios are markers for neurodegeneration due to abnormal metabolism, including a decreased N-acetylaspartate (NAA)/creatine ratio, increased choline/creatine ratio, and increased lactate/creatine ratio in the striatum HD patients (Jenkins et al., 1998). By detecting metabolite ratio abnormalities in HD patients compared to normal ratio levels of non-HD age matched controls, changes in ratios can be assessed before, during, and after treatment. An improvement in cellular metabolism would be seen by the return of metabolite ratios closer to or back to normal levels.

The term “standardized scales” refers to questionnaires and inventories to assess an individual with Huntington's disease, but are not limited to, the Total Functional Capacity (TFC) portion of the Unified Huntington's Disease Rating Scale (UHDRS) and the mini-mental state examination (MMSE).

The term “Total Functional Capacity (TFC) portion of the Huntington's Disease Rating Scale (UHDRS) as used herein, refers to, a scale used to assess the degree of disease progression characterized by a numerical value, a score of 13 represents a normal degree of function while a score of 0 represents a severely disabled state (Huntington Study Group, 1996).

The term “mini-mental state examination (MMSE) ” refers to a scale used to assess the degree of cognitive impairment and measure changes in cognitive impairment over time characterized by a numerical value. A score of 30 represents the highest score and normal cognition and a score of 0 represents the lowest score and very severe cognitive impairment (Folstein et al., 1975).

### Methods of Treating Huntington's Disease

The present invention includes use of a therapeutically effective amount of tissue kallikrein or a variant or active fragment thereof in the treatment of the prodrome and onset stage of Huntington's disease, juvenile Huntington's disease, or symptoms thereof. In an embodiment of the invention, tissue kallikrein, or a variant or active fragment thereof, may be administered concurrently with a second therapeutic compound useful in treating Huntington's disease. Examples of compounds useful in the treatment of Huntington's disease are discussed in greater detail below. A therapeutically effective amount of tissue kallikrein or a variant or active fragment thereof may be administered orally or more preferably, intranasally. Methods of administration are discussed in greater detail below.

The present invention further provides use of tissue kallikrein or a variant or active fragment thereof in treating conditions associated with Huntington's disease including minor motor skill abnormalities of restlessness, abnormal eye movements, hyperreflexia, impaired finger tapping, rapid alternating hand movements and mild dysarthria (speech); involuntary motor abnormalities such as chorea (rapid, ceaseless movements) bradykinesia, hypokinesia, rigidity and dystonia; voluntary motor impairments such as dysphagia (swallowing), dysarthria, and gait disturbances; muscle wasting, dehydration, and weight loss; non-motor symptoms of cognitive deficits in concentration, organization, spatial perception, memory skills (dementia), and non-cognitive psychiatric deficits of depression (low energy, sleep disturbances), personality changes (irritability, low energy, apathy anhedonia) and bipolar disorder (delusions, hallucinations, paranoia).

The prodrome stage of Huntington's disease is a progressive neuronal hypometabolism and neurodegeneration (neuronal cell loss/brain atrophy/volume loss) of the striatum, cerebral cortex, and white matter areas of the brain as detected by PET and MRI prior to adult onset of HD. The prodrome stage of HAD is thought to be an ongoing process 10-20 years before adult onset with gradually changes in motor skill, behavioral, and psychological areas being seen.

Another embodiment of the invention includes the use of a therapeutically effective amount tissue kallikrein or a variant or active fragment thereof for treating the prodrome stage of Huntington's disease.

Juvenile Huntington's disease is classified as those under the age of 20 years when Huntington's disease appears. The juvenile form tends to have 50 or more CAG repeats in the mutant HD gene, with the higher the number of repeats the earlier the onset of the disease. The early the onset, the more likely rigidity is to be seen and less likely to show chorea movements. A unique feature in juvenile Huntington's disease is the tendency of epileptic seizures, not seen in adult onset HD. Children will the disease will show gradual changes in behaviour and cognitive function.

Another embodiment of the invention includes the use of a therapeutically effective amount tissue kallikrein or a variant or active fragment thereof for treating Juvenile Huntington's disease.

Huntington's disease is associated with brain-specific abnormalities in transcription, BDNF survival signaling, ubiquitin-proteasome degradation mitochondrial metabolism and ROS detoxifying enzymes, axonal transport, glutamate-NMDA receptor signaling and caspase activation due to the expression of mutant huntingtin protein. In transgenic R6/2 mice, which express the N-terminal fragment of the human HD gene with 150 CAG repeats, neurological symptoms that resemble many of those seen in HD develop including a decline in motor co-ordination, altered locomotor activity, impaired cognitive performance, seizures and significant atrophy of the striatum and cortex areas of the brain due to neurodegeneration (Turmaine et al., 2000; Zourlidou et al., 2007). R6/2 mice display impaired neurogenesis in the dentate gyrus region of the hippocampus (Phillips et al., 2005), such that neurodegeneration repair is likely impaired. Nerve growth factor (NGF) is a neurotrophic factor which promotes neurogenesis in the hippocampus (Frielingsdorf et al., 2007). An increase in neurogenesis may result in neuronal migration to areas of degeneration in the brain and repair the damage, and reverse atrophy leading to improved volume as detected by MRI scans. Another embodiment of the invention includes the use of a therapeutically effective amount tissue kallikrein or a variant or active fragment thereof for improving volume in the brain of a HD patient.

Huntington's disease patients display a reduced ability to uptake glucose in areas of the cortex and basal ganglia (which includes the striatum), and can be linked to the abnormal cellular metabolism detected in the HD brain, including decreased N-acetylaspartate levels (NAA/creatine ratio) and increased choline levels (choline/creatine ration) both markers of neurodegeneration and increased levels of lactate. Dysregulation of transcription by mutant Huntingtin, leads to abnormal cellular metabolism as the loss of ROS-detoxifying enzymes and mitochondrial biogenesis results in mitochondria dysfunction. This results in less energy ATP production and increased ROS oxidative stress to the cell.

Another embodiment of the invention includes a method of improving glucose uptake and cellular metabolism by the brain of a patient comprising administering a therapeutically effective amount of tissue kallikrein or a variant or active fragment thereof.

Another embodiment of the invention includes a method for improving improvement blood flow and/or oxygen uptake to the brain of a patient comprising administering a therapeutically effective amount of tissue kallikrein, or a variant or active fragment thereof.

### Administration of Tissue Kallikrein

Traditional modes of drug administration to treat aliments in the brain include oral as well as intravenous routes of administration. These modes are not always ideal. Oral administration of compounds results in limited bioavailability (solubility, 1^{st} pass liver degradation, blood brain barrier restriction) as well as time release issues with potentially undesirable gastrointestinal side effects. However, tissue kallikrein (KLK1) appears able to pass through and may bypass the blood-brain-barrier to produce its effects on the brain.

Intravenous (i.v.) administration requires trained medical professionals, which is time consuming and costly to the health care system. It may also result in patient compliance issues. Risks associated with intravenous administration, include infection at the injection site and safety issues to both the patient and the professional administering the dose. However, in a controlled setting, intravenous administration can be effective.

Intranasal administration allows a medicament to be ‘fast acting’ since it is able to reach the brain by a more direct route. Intranasal administration is convenient and virtually eliminates issues of patient compliance as seen with intravenous administration. Olfactory epithelial cells are selectively permeable. Thus, proteins such as KLK1 can pass through and may bypass the blood-brain-barrier via the intranasal route. Thereby intranasal administration of KLK1 may produce its effects directly on the brain—thereby minimizing peripheral effects as well. This is due to involvement of the olfactory region in the upper portion of the nasal pathway.

There are two possible routes that a substance administered intranasally may follow at the olfactory region—intraneuronal and extraneuronal. An intraneuronal route includes uptake of peptides into olfactory neurons where peptides travel along axons to bypass the blood-brain-barrier. Passage through unique intercellular clefts in epithelia of the olfactory region is an extracellular route that allows peptides to diffuse into the subarachnoid space. An extracellular route is more preferable due to rapid passage time to the brain, avoidance of proteolytic degradation involved in intraneuronal pathways (Born et al., Nat. Neurosci. 2002, 5(6):514-6), and rapid eliciting of biological effects at multiple sites of the brain (Throne et al., 2004).

Intranasal administration can provide an advantage over oral administration by more direct delivery of KLK1 to desired sites of action (the brain).

Pharmaceutical compositions of the invention may be administered orally, intravenously, or intranasally. Formulations suitable for intranasal administration include ointments, creams, lotions, pastes, gels, sprays, aerosols, oils and the like. Solutions or suspensions are applied directly to the nasal cavity by conventional means, for example, with a dropper, pipette, or spray. Formulations may be provided in a single or multidose form. In the latter case of a dropper or pipette, this may be achieved by the patient administering an appropriate, predetermined volume of the solution or suspension. A spray includes a metering atomizing spray pump.

An active ingredient for an aerosol formulation can be provided in a pressurized pack with a suitable propellant including, but not limited to, a chlorofluorocarbon (CFC), dichlorodifluoromethane, trichlorofluoromethane, or dichlorotetrafluoroethane, or carbon dioxide or other suitable gas. An aerosol may also contain a surfactant such as lecithin. A dose of drug may be controlled by a metered valve. Alternatively active ingredients may be provided in a form of a dry powder. A powder mix of the compound can be in a suitable powder base such as lactose, starch, or starch derivatives such as hydroxypropylmethyl cellulose and polyvinylpyrrolidine (PVP). The powder carrier can form a gel in the nasal cavity. A powder composition may be presented in unit dose form, including, but not limited to, capsules or cartridges (e.g., gelatine or blister packs from which the powder may be administered by means of an inhaler).

Oral administration includes enteral administration of solution, tablets, sustained release capsules, enteric coated capsules, orally disintegrating tablets and syrups.

An “effective amount” or a “therapeutically effective amount” refers to a nontoxic but sufficient amount of drug or agent to provide a desired effect. In a combination therapy, an “effective amount” of one component of the combination is an amount of that compound that is effective to provide a desired effect when used in combination with the other components of the combination. An amount that is “effective” will vary from subject to subject, depending on the age and general condition of an individual, a particular active agent or agents, and the like. An appropriate “effective” amount in any individual case may be determined using routine experimentation.

A therapeutically effective amount of a compound of the invention for treating the above-identified diseases or symptoms thereof can be administered prior to, concurrently with, or after the onset of the disease or symptom. A compound of the invention can be administered concurrently with the onset of the disease or symptom. “Concurrent administration” and “concurrently administering” as used herein includes administering a polypeptide of the invention and another therapeutic agent in admixture, such as, for example, in a pharmaceutical composition or in solution, or separately, such as, for example, separate pharmaceutical compositions or solutions administered consecutively, simultaneously, or at different times, but not so distant in time such that the compound of the invention and the other therapeutic agent cannot interact and a lower dosage amount of the active ingredient cannot be administered.

Another aspect of the present invention includes a method as described herein further comprising concurrently administering an additional therapeutic compound useful in treating the prodrome and onset stage of Huntington's disease, juvenile Huntington's disease or symptoms thereof. An additional Huntington's disease therapeutic compound includes, but is not limited to, a nutritional supplement, an anti-oxidant, an anti-apoptotic compound, a NDMA receptor antagonist, a mitochondrial stabilizer, a dopamine blocker, a BDNF inducer, an inducer of transcription, a blocker of mutant huntingtin allele expression, a neurotrophic factor, inducer of autophagy, or restorative neural stem cell therapy. A nutritional supplement can be CoQ10, Ethyl-EPA, or creatine; an anti-oxidant can be cysteamine, indole-3-propionic acid, N-acetylcysteine, glutathione, or bucillamine; an anti-apoptotic compound can be minocycline, tauroursodexoycholic acid, methazolamide, or a caspase-6 inhibitor; an NDMA receptor antagonist can be memantine or remacemide; a mitochondrial stabilizer can be Dimebon; a dopamine blocker can be tetrabenazine; a BDNF inducer can be cysteamine or Citalopram; a transcription inducer can be a HDAC inhibitor; a blocker of mutant huntingtin allele expression can be a huntingtin gene specific RNAi or antisense molecule; a neurotrophic factor can be FGF2 or Neurturin; an inducer of autophagy can be verapamil, clonidine, or rampamycin; a restorative neural stem cell therapy can be introducing isolated stem cells into the brain; an antipsychotic can be haloperidol, chlorpromazine, or olanzapine; an antidepressant can be fluoxetine, sertraline hydrochloride, or nortriptyline; a tranquilizer can be benzodiazepine, paroxetine, venlafaxin or beta-blockers; a mood-stabilizer can be lithium, valproate or carbamazepine; and a muscle relaxant can be Botulinum toxin.

“Treatment” and “treating” refer to inhibiting and/or alleviating a disease and related symptoms as well as healing disease conditions or symptoms affecting mammalian organs and tissues. “Prevention” and “preventing” refer to keeping the disease from occurring. A composition of the present invention can be administered in a therapeutically effective amount to a patient before, during, and after any mentioned condition arises.

### Pharmaceutical Compositions

The present invention provides pharmaceutical compositions comprising tissue kallikrein, or a variant or active fragment thereof suitable for oral and intranasal administration in the treatment of the prodrome and adult onset stage of Huntington's disease, Juvenile Huntington's disease and symptoms thereof.

In one aspect, the present invention provides a pharmaceutical composition comprising about 0.001 to about 1000 International Units (IU) per dosage frequency of tissue kallikrein, or a variant or active fragment thereof, and a pharmaceutically acceptable excipient formulated for oral administration. An intranasal dose of tissue kallikrein, or a variant or active fragment thereof can be about 0.001 to 100 IU. An intranasal dose of tissue kallikrein, or a variant or active fragment thereof can be about 0.001 to 10 IU. An intranasal dose of tissue kallikrein, or a variant or active fragment thereof can be about 0.01 to 10 IU. An intranasal dose of tissue kallikrein, or a variant or active fragment thereof can be about 0.01 to 1 IU. An intranasal dose of tissue kallikrein, or a variant or active fragment thereof can be about 0.1 to 1 IU.

In another aspect, the present invention provides a pharmaceutical composition comprising about 0.001 to about 5000 IU per dosage frequency of tissue kallikrein, or a variant or active fragment thereof, and a pharmaceutically acceptable excipient formulated for intranasal administration. An oral dose of tissue kallikrein, or a variant or active fragment thereof can be about 0.001 to 500 IU. An oral dose of tissue kallikrein, or a variant or active fragment thereof can be about 0.001 to 50 IU. An oral dose of tissue kallikrein, or a variant or active fragment thereof can be about 0.01 to 50 IU. An oral dose of tissue kallikrein, or a variant or active fragment thereof can be about 0.01 to 5 IU. An oral dose of tissue kallikrein, or a variant or active fragment thereof can be about 0.1 to 5 IU. An oral dose of tissue kallikrein, or a variant or active fragment thereof can be about 0.1 to 1 IU.

The pharmaceutical composition may further comprise a second therapeutic compound useful in treating the prodrome and adult onset stage of Huntington's disease, or Juvenile Huntington's disease as discussed above.

Pharmaceutical compositions of the invention include formulations to be administered orally, intravenously, or intranasally. Formulations suitable for intranasal administration include powder, granules, solution, drops, ointments, creams, lotions, pastes, gels, sprays, aerosols, oils and the like. Solutions or suspensions of the invention can be applied directly to the nasal cavity by conventional means, for example, with a dropper, pipette, or spray. Formulations may be provided in a single or multidose form. A solution may be sterile, isotonic, or hypotonic, and otherwise suitable for administration by injection or other means and may contain appropriate adjuvants, buffers, preservatives, and salts. Solutions such as nose drops may contain antioxidants, buffers, and the like. Powder or granular forms of a pharmaceutical composition can be combined with a solution and with diluting, dispersing, and/or surface active agents.

Formulations for aerosol administration include formulations designed for intranasal administration. An active ingredient can be provided in a pressurized pack with a suitable propellant such as a chlorofluorocarbon (CFC) (e.g., dichlorodifluoromethane, trichlorofluoromethane, or dichlorotetrafluoroethane), carbon dioxide, or other suitable gas. An aerosol may also contain a surfactant such as lecithin. A dose of drug may be controlled by a metered valve. Alternatively active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. A powder composition may be presented in unit dose form for example in capsules or cartridges of e.g., gelatine or blister packs from which the powder may be administered by means of a device.

A pharmaceutical composition formulated for intranasal administration comprises about 0.001 to about 5000 IU of KLK1, or a variant, or an active fragment thereof, optionally, further comprising a pharmaceutically acceptable excipient. Formulations suitable for oral administration include liquids, pills, solution, tablets, sustained release capsules, enteric coated capsules or syrups. A pharmaceutical composition formulated for oral administration comprises about 0.001 to 1000 IU of KLK1, or a variant or an active fragment thereof, optionally further comprising a pharmaceutically acceptable excipient.

### Pharmaceutical Compositions Useful for Intranasal Administration and Uses Thereof

An aspect of the invention includes a composition formulated for intranasal administration comprising about 0.001 to about 5000 IU of KLK1, or a variant or an active fragment thereof, optionally comprising a pharmaceutically acceptable excipient.

A composition can be administered to the nasal cavity of a human or other mammal to diseased areas of the brain by means of the olfactory neural pathway. The method may employ a pharmaceutical composition capable of transporting KLK1 to diseased neurons of the brain.

A method of the invention can deliver of compounds to afflicted areas of the brain through transneuronal retrograde and anterograde transport mechanisms. Delivery of neurologic agents to the brain by that transport system can be achieved in several ways. One technique comprises delivering a neurologic agent alone to the nasal cavity. In this instance, chemical characteristics of KLK1 can facilitate its transport to diseased neurons in the brain. Peripheral nerve cells of the olfactory neural pathway can be utilized in order to deliver KLK1 to damaged neurons in those regions of the brain that are connected to the olfactory bulb.

KLK1 can be administered to the nasal cavity alone or in combination with a second therapeutic compound useful in treating the prodrome and adult onset stage of Huntington's disease, and Juvenile Huntington's disease. KLK1 can be combined with a carrier and/or other adjuvants to form a pharmaceutical composition. Potential adjuvants include, but are not limited to, GM-1, phosphatidylserine (PS), and emulsifiers such as polysorbate 80. Further supplementary substances include, but are not limited to, lipophilic substances such as gangliosides and phosphatidylserine (PS).

A method of the invention delivers KLK1 to the nasal cavity of a mammal. It is preferred that KLK1 be delivered to the olfactory area in the upper third of the nasal cavity and particularly to the olfactory epithelium to promote transport of the agent into the peripheral olfactory neurons rather than the capillaries within the respiratory epithelium. Thereby KLK1 is transported by means of the nervous system to the brain and damaged neurons in the brain.

In one embodiment of the method of the invention, KLK1 can be combined with micelles comprised of lipophilic substances. Such micelles can modify the permeability of the nasal membrane and enhance absorption of the agent. Lipophilic micelles include gangliosides, particularly GM-1 ganglioside, and phosphatidylserine (PS).

Once KLK1 has crossed the olfactory epithelium, the invention further provides transport of KLK1 along the olfactory neural pathway. KLK1 is capable of movement within the olfactory system. In particular, neurotrophic and neuritogenic substances have demonstrated ready incorporation into nerve cell membranes and an affinity for nerve cell receptor sites.

To deliver KLK1 to olfactory neurons, KLK1 alone or in combination with other substances as a pharmaceutical composition can be administered to the olfactory area located in the upper third of the nasal cavity. A composition can be dispensed intranasally as a powdered or liquid nasal spray, nose drops, a gel or ointment, through a tube or catheter, by syringe, by packtail, by pledget, or by submucosal infusion.

A pharmaceutical composition for intranasal administration may be formulated as a powder, granules, solution, ointment, cream, aerosol, powder, or drops. A solution may be sterile, isotonic or hypotonic, and otherwise suitable for administration by injection or other means. In addition to KLK1, a solution may contain appropriate adjuvants, buffers, preservatives and salts. Powder or granular forms of a pharmaceutical composition may be combined with a solution and with diluting, dispersing and/or surface active agents. Solutions such as nose drops may contain antioxidants, buffers, and the like.

The olfactory system provides a direct connection between the outside environment and the brain thus providing quick and ready delivery of KLK1 for treating the prodrome and adult onset stage of Huntington's disease, Juvenile Huntington's disease and amnesiac mild cognitive impairment. Moreover, means of applying a pharmaceutical composition intranasally can be in a variety of forms such as a powder, spray, or nose drops that obviates intravenous or intramuscular injections and simplifies administration of therapeutic medications.

The invention will be described with reference to various specific and preferred embodiments and techniques. However, it should be understood that many variations and modifications may be made while remaining within the spirit and scope of the invention.

### EXAMPLES

### Example 1

### Prevention of Huntington's Disease in R6/2 Transgenic Mouse Model After Treatment with KLK1

Administration of KLK1 improved behavioral deficits, body weight loss, and survival in the R6/2 transgenic mouse model of Huntington's disease (HD).

### Methods

Forty Five male and female R6/2 transgenic (TG) mice and 15 male and female wild-type littermates (Jackson Labs, USA) were used in the experiment. Animals were housed at a standard temperature (22±1° C.) in a light-controlled environment (lights on from 7 am to 8 pm) with ad libitum access to food and water. The animals were divided into the following treatment groups:

1) 15 wild-type littermate control mice treated with PBS Vehicle (24 μl), intranasally, starting at 4 weeks of age and continuing daily until end-point at 20 weeks of age.

2) 15 R6/2 TG mice treated with PBS Vehicle (24 μl), intranasally, starting at 4 weeks of age & continuing daily until end-point at 20 weeks of age

3) 15 R6/2 TG mice treated with 5 mg/ml of KLK1 in 24 μl PBS, intranasally, starting at 4 weeks of age & continuing until end-point at 20 weeks of age

4) 15 R6/2 TG mice treated with 50 mg/ml of KLK1 in 24 μl PBS, intranasally, starting at 4 weeks & continuing until end-point at 20 weeks of age

The general health status of each animal was monitored on a daily basis, and an animal was sacrificed if its health significantly worsened, including no spontaneous movements and inability to drink or eat in 24-h observation period, massive bleeding, spontaneous inflammation, missing anatomy, swelling, or tumors.

### Body Weight and Survival

Body weight was measured and recorded at the start of the study (4 weeks of age) and then two times a week until end of the study at twenty weeks of age. Additionally, the mice were observed twice-a-day for mortality and results were recorded.

### Motor Skills Testing:

### Rotarod

The mice were tested to assess motor coordination by placing the animals on a rotating rod (TSE Systems, Germany). The test was performed for each group at 4, 6, 8, 10, and 12 weeks of age, and mice were tested in two consecutive days. Mice were first individually exposed to the apparatus for a 5 min training session at a constant speed of 4 rpm. If a mouse fell off the rotating rod during training it was placed back on the rod until the 5 min elapses. The training session was followed by a 1-hour rest period back in their cage. After a 1-hour rest, mice were individually tested on the rotarod apparatus for two trials at an accelerating speed (0-40 rpm) over 5 minutes. Each trial was separated by a 30-minute rest period. Each time a mouse fell off the rotarod, the latency to fall and the speed of rotarod at the time of a fall was recorded.

### Grip Strength

Grip strength measurements were performed once-a-week at 4, 6, 8, 10, and 12 weeks of age using an Ugo Basile machine (Ugo Basile, Italy) for each group. A mouse was placed on the grip-strength apparatus and forced to grab a small triangular handle with its forepaws. Next, the test mouse was lowered to the platform and then slowly pulled away from the handle by the tail until the test mouse released the handle with its forepaws. This process was repeated, five scores were recorded per mouse in consecutive sequence.

### Open Field

Open field tests were performed at 4, 6, 8, 10, and 12 weeks of age for each group. Activity chambers (Med Associates Inc, St Albans, Vt.; 27×27×20.3 cm) were equipped with IR beams and mice were placed in the center of the chamber and their behavior was recorded for 15 min in 5-minute bins. Quantitative analysis was performed on the measures total locomotion and rearing frequency.

### MRI and MRS Analysis

MRS and MRI analysis was performed for all mice at the age of 12 weeks in a horizontal 4.7 T magnet with bore size 30 cm (Magnex, Abington, UK) equipped with Magnex gradient set (max. gradient strength 170 mT/m, bore 12.5 cm) interfaced to a Varian UNITYINOVA console (Varian, Inc., Palo Alto, Calif.) using a quadrature half volume coil (coil diameter 16 mm, HF Imaging LLC, Minneapolis, Minn., USA) for transmission and reception. Isoflurane-anesthetized mice were fixed to a head holder and positioned in the magnet bore in a standard orientation relative to gradient coils. For determination of volume of brain, striatum, and lateral ventricles, T2-weighted multi-slice (12-14 continuous slices) images were acquired using double spin-echo sequence with adiabatic refocusing pulses TR=2 s, TE=65 ms, matrix size of 256×128, FOV of 20*20 mm2, and a slice thickness of 0.7 mm, 4 averages.

1H-MRS data were collected using the same experimental setup. Voxel of 3×3×3 mm³ was placed in the striatum of the mouse based on T2-weighed images collected as described above. Fast, Automatic Shimming Technique by Mapping Along Projections (FASTMAP) is used to adjust BO homogeneity in the voxel. The water signal was suppressed using variable power RF pulses with optimized relaxation delays (VAPOR) to obtain B1 and T1 insensitivity. A short echo time STEAM sequence (TE=2, TM=30 ms) combined with outer volume suppression (OVS) was used for the pre-localization. Three OVS blocks were used interleaved with water suppression pulses. Data were collected by averaging 256 excitation with TR of 4s, number of points 2048 and spectral width 2 kHz. In addition a reference spectrum without water suppression was collected from the identical voxel using the same acquisition parameters. Peak area for major metabolites (NAA, Cho, Tau, m-INS, Glu, Gln, GABA, Cr, PCr and Glx) were analyzed using LC-model and results were given as absolute values.

### Neurogenesis

To detect neurogenesis, the cell proliferation specific marker 5-bromodeoxyuridine (BrdU, Sigma) was administered repeatedly (50 mg/kg BrdU dissolved in saline, i.p., 5 ml/kg). BrdU injections were started at week 18 and continued once-a-day for 14 days (until end-point at week 20).

At 20 weeks of age, mice in all groups were sacrificed and brains were removed from the skull. The right brain hemisphere was fixed by immersion in 4% PFA in phosphate buffer for 24 h. After cryoprotection in 30% sucrose for 2-3 days and freezing in liquid nitrogen, the right hemisphere was cut in 20-μm-thick coronal cryosections with a cryostat (Microm) and collected on SuperFrost Plus glass slides. Four glasses were collected starting from dorsal hippocampal level. The hippocampal sections covered dentate gyrus and subventricular zone (SVZ). The anti-BrdU immunohistochemistry was performed with a standard IHC protocol. Briefly, the sections were re-hydrated by a 5-min incubation in 0.1 M PBS for 5 min and treated with 2×SSC/50% formamide at 65° C. for 2 h followed by incubations in 2×SSC for 5 min, in 2M HCl for 30 min, and 0.1M borate buffer for 30 min to expose the antigen. After washes in 0.1M PBS, the activity of endogenous peroxidases was blocked by incubation in 0.3% H2O2 in methanol. After three 5 min washes in 0.1 M PBS containing 0.05% Tween®-20 (PBST), the sections were blocked with 10% NHS in PBST for 30 min at room temperature. The sections were washed and incubated in primary antibody (anti-BrdU) solution diluted 1:300 in 5% normal horse serum (NHS) in PBST overnight at room temperature. After three 5 min washes in PBST, the sections were incubated in secondary antibody solution (biotinylated horse anti-mouse IgG, 1:200 in 5% NHS) for 2 h at room temperature. The sections were washed followed by incubation in avidin-biotin HRP complex (Vector Vectastain ELITE kit) for 2 h at room temperature. The immunoreaction was visualized using Ni-enhanced DAB as a substrate (Vector DAB kit).

Unbiased stereologic analysis was applied to count total numbers of cells that are immunopositive for BrdU. The designated areas were outlined using CFI Plan Achro 4× objective. Counts were performed using CFI Plan Achro 100× oil immersion objective (N.A. 1.30, W.D. 0.20) on Eclipse E600 microscope with a 3-chip CCD color camera and

LEP motorized stage (3-axis computer controlled stepping motor system with a 0.1-μm resolution). Total cell numbers were estimated using Stereo Investigator 7 software, the optical fractionator method (MicroBrightField, Colchester, Vt.). The software randomly overlays a sampling grid area of 14,400 μm² (hippocampus) and 8100 μm² (SVZ) on top of the drawn contour. The counting frame was 900 μm² for cell countings. The mean thickness of the mounted sections was 20 μm. The plane for the top of the counting frame in the z-dimension was set to 5 μm from the surface of the section (guard zone). The counting criterion was that the top of the BrdU-positive nucleus come into focus within the dissector height.

### Results

Survival. Treatment with KLK1 extended survival time for mice in group 3 and 4. The two groups showed a statistically significant increase in survival time compared to the non-treated group 2.

Motor Performance. The motor performance of group 3 and 4 was improved as the latency time to falling over the rotarod increased in these groups treated with KLK1.

The latency to fall improvement in group 3 and 4 was statistically significant in comparison to group 2. Group 3 and 4 also displayed an increase in grip strengthen (peak tension, measured in kg) compared to the non-treated group 2, which was statistically significant.

In open field testing, group 3 and 4 showed increased total locomotion and rearing frequency scores compared to group 2. This increase in mice treated with KLK1 was found to be statistically significant.

Brain volume and metabolism. Group 3 and 4 showed a significant increase in brain volume as measured by MRI in comparison to the untreated group 2. This suggests that KLK1 treatment helps to prevent brain atrophy seen in the R6/2 HD transgenic model, in areas such as the striatum and cortex of the brain. Additionally, analysis by MRS showed an improvement in cellular metabolism in group 3 and 4 mice treated with KLK1 compared to group 2. The difference was significant as group 3 and 4 were closer to the normal levels of NAA/creatine seen in the control group 1 then the non-treated group 2, indicative of improved cellular metabolism in groups 3 and 4. This result helps to explain the increase of brain volume seen by MRI studies, as neurodegeneration has decreased as indicated by an improved NAA/creatine ratio.

### Neurogenesis

Group 3 and 4 had a statistically significant increase in BrdU positive cells of the hippocampal dentate gyrus and subventricular zone (SVZ) regions in comparison to the non-treated group 2. This result suggests that KLK1 treatment stimulates neurogenesis and therefore helps to counter neurodegeneration seen in the R6/2 model overtime.

1. A method of treating Huntington's Disease or symptoms thereof, or juvenile Huntington's disease or symptoms thereof, comprising administering tissue kallikrein or a variant or active fragment thereof.

2. The method of claim 1 wherein the treatment is of a prodrome and onset stage of Huntington's disease or symptoms thereof.

3. The method of claim 1, wherein the tissue kallikrein is human.

4. The method of claim 1, wherein the tissue kallikrein is SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, or a variant or active fragment thereof.

5. The method of claim 1, wherein the tissue kallikrein, variant, or active fragment thereof has 80% identity with SEQ ID NO: 1.

6. The method according to claim 1, wherein the tissue kallikrein is an isolated form, a synthetic form, or a recombinant form of tissue kallikrein.

7. The method according to claim 1, wherein the tissue kallikrein is administered concurrently with a second therapeutic compound useful in treating Huntington's disease or juvenile Huntington's disease.

8. A method of treating Huntington's Disease or symptoms thereof, or juvenile Huntington's disease or symptoms thereof comprising administering a composition comprising tissue kallikrein or a variant or active fragment thereof, and a pharmaceutically acceptable carrier.

9. The method of claim 8 wherein the treatment is of a prodrome and onset stage of Huntington's disease or symptoms thereof.

10. The method of claim 8, wherein the pharmaceutical composition comprises about 1 to about 1000 IU per day of tissue kallikrein, or a variant or active fragment thereof.

11-18. (canceled)
